(19) United States
(12) Patent Application Publication
(10) Pub. No.: US 2005/0163686 A1
(43) Pub. Date: Jul. 28, 2005

(54) Sterilization case with matrix base

(75) Inventors: Cary A. Bettenhausen, Indianapolis, IN (US); Todd E. Bettenhausen, Indianapolis, IN (US)

Correspondence Address: Frank D. Lachenmaier;Suite 200, 116 N. Main St., P.O. Box 1047, Kokomo, IN, 46903-1047, US

(73) Assignees: William I. Summers, Kokomo, IN (US); Connie L. Summers, Kokomo, IN (US)

(21) Appl. No.: 11/037,634

(22) Filed: Jan. 18, 2005

Related U.S. Application Data
(60) Provisional application No. 60/536,657, filed on Jan. 15, 2004.

(51) Int. Cl. A61L 2/26; A61L 2/16

(52) U.S. Cl. 422/292; 422/300; 206/370

(57) ABSTRACT

In accordance with the present invention a tray for sterilizing, storing, transporting and presenting surgical instruments is formed in a matrix format by molding, extrusion or assembling a matrix of interlocking egg crate type members with the contours of the instruments in question and in some cases a basket shape to hold several small instruments such as screws machined into the top surface of the matrix. The matrix is light, strong and burr free and is an open structure facilitating the passage of the sterilizing media, steam, past the instruments in question.

### BACKGROUND

1. Field of Invention:

This invention relates generally to materials handling apparatus and an improved method of manufacture of same. More specifically, this invention relates to an integrated system of pockets and baskets for facilitating sterilization, storage and presentation of surgical instruments.

1. Prior Art:

A variety of sterilization cassettes exist with different shapes and styles of pockets and retainers to hold various surgical instruments securely during sterilization. Where possible, some surgical instruments are single use, arriving from the manufacture in a sterile package to be opened during surgery and then thrown away. There are many instruments used in modern surgery, however, that are delicate, complex and expensive and must be sterilized and re-used. A common practice is also to present all of the instruments to be used in a given surgery to the surgeon in a common cassette or carrier. Holding the instruments securely during sterilization and transport is usually accomplished by sandwiching them between a deformable member with sterilant ingress ports and a top enclosure with mating sterilant ingress ports. Once a pre-arranged cassette is loaded with the correct instruments for the desired surgery and sterilized, the cassette is typically wrapped in a sterile gauze material and stored until required transportation to the next operating room. It is unwrapped, the top enclosure and deformable member removed and presented to the surgical team. These sterilization trays suffer from handling damage and relatively quick obsolescence and are expensive to maintain.

The myriad of such cassettes or carriers can be broken into two major types each with their own advantages and drawbacks. The first and earliest of these were sheet metal trays with brackets riveted into holes stamped onto the bottom of the tray and weep ports punched through the bottom of the sheet metal tray to allow for egress of the sterilizing condensate. Side walls are formed up from the bottom blank, overlapped and riveted forming a secure lower box. An example of this type is shown in FIG. 1 PRIOR ART. The brackets are sized to fit the instruments required for a given operation. Sheet metal cases have a relatively high coefficient of heat transfer that allowed fairly rapid drying, even though the drying cycle is still the longest segment of the sterilization process. Rapid drying may be critical if an instrument is dropped during an operation and requires re-sterilization before the operation can be completed. Some kits compensate for this possibility by including redundant instruments within the kit at the obvious extra cost.

The two biggest drawbacks to the stamped sheet metal case are the inherent weight and the fact that it very difficult and expensive to punch holes in sheet metal with conventional punch and die clearance leaving no burrs or sharp edges around the openings or edges of tray. Some instruments are fairly large and heavy and their packaging is limited by the weight that the technician can handle safely. As mentioned above, once the tray full of instruments has been sterilized, it is desirable to have it wrapped in sterile gauze and stored awaiting transport to an operating room and presentation to the surgical team. Any sharp edges or corners can pierce the sterile gauze and require re-sterilization before use

A number of cassettes have been fabricated from high temperature plastics molded or vacuum formed in an attempt to reduce these two problems. The plastic cases are inherently lighter in weight and can be molded or formed essentially burr and sharp edge free. An example of this type is shown in FIG. 2 PRIOR ART.

Continued exposure to high temperatures and humidity, essentially an autoclave environment, however, can cause deterioration and fracturing of some plastics over time. The biggest problem with the plastic carriers, however, is the relatively low coefficient of heat transfer. As mention above, the drying time is dramatically impacted by the case's ability to conduct the heat away from the instruments.

### SUMMARY

An object of the present invention is to provide a container for sterilizing, storing, transporting and presenting surgical instruments of different sizes and shapes in a lightweight cassette with a high heat transfer coefficient and formed with no sharp edges or burrs. Another object of the present invention is to provide a cassette that retains the instruments securely during processing, but allows the surgical team to remove them readily when required. A further object is for the instrument retaining case to be economical to revise or replace, facilitating different instruments when the procedure changes, the sequence of presentation changes or the tray is damaged in handling. The case should also contain basket shaped pockets to hold small instruments such as screws. The sterilization case is made in a vertically oriented matrix format and can be formed by-molding, extrusion or as a combination of interlocking rectangular members. The matrix can have as many or as few members as the individual surgical procedure demands and can be made of various materials. The matrix can have the non-weight bearing portions of the interlocking members removed except for the structural connections resulting in strong lightweight load carrying structures.

### DRAWINGS

In order that the invention may be more fully understood it will now be described by way of example, with reference to the accompanying drawings in which:

FIG. 1 is a front perspective view of a Prior Art stamped sheet metal case with riveted brackets.

FIG. 2 is a front perspective view of a Prior Art molded plastic sterilization case.

FIG. 3 is a front perspective view of a molded or extruded matrix sterilization case.

FIG. 4 is an exploded view of an interlocking member matrix sterilization case.

FIG. 5 is an exploded view of an interlocking member matrix sterilization case with various components required for the several applications shown in phantom lines for illustrative purposes and not part of this invention

### REFERENCE NUMERALS

The same reference numbers are used to refer to the same or similar parts in the various views.
10—Instrument sterilization case
12—pockets
14—instruments
16—weep or egress port
18—brackets
20—top enclosure
22—deformable member
24—filter
26—ingress ports
28—outer wrapper
30—bottom interlock member
32—top interlock member
34—hold down interlock member

### DESCRIPTION

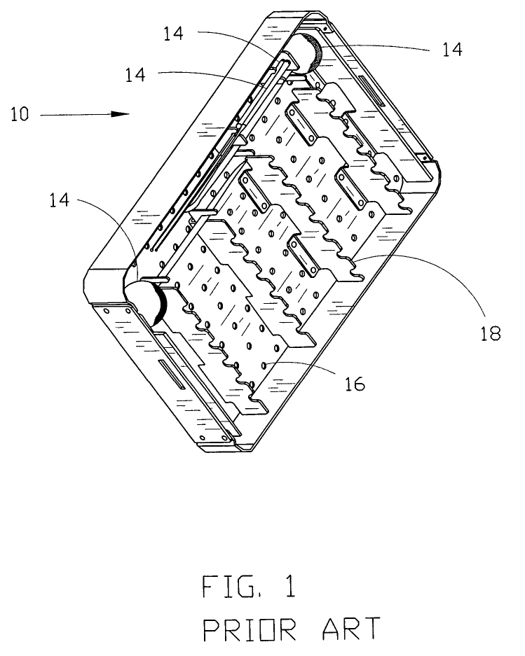

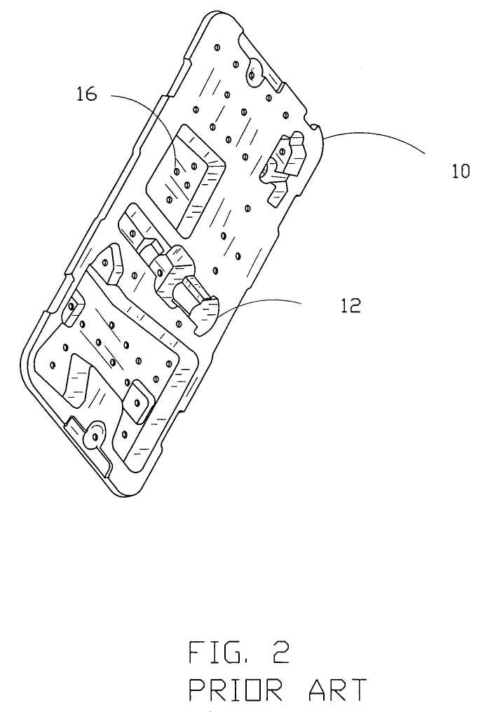

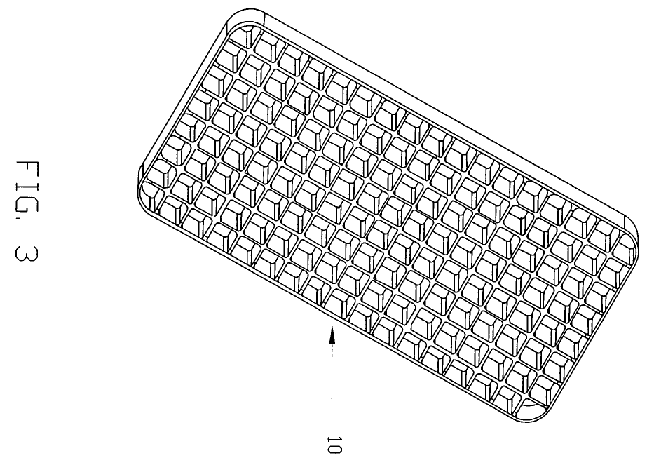

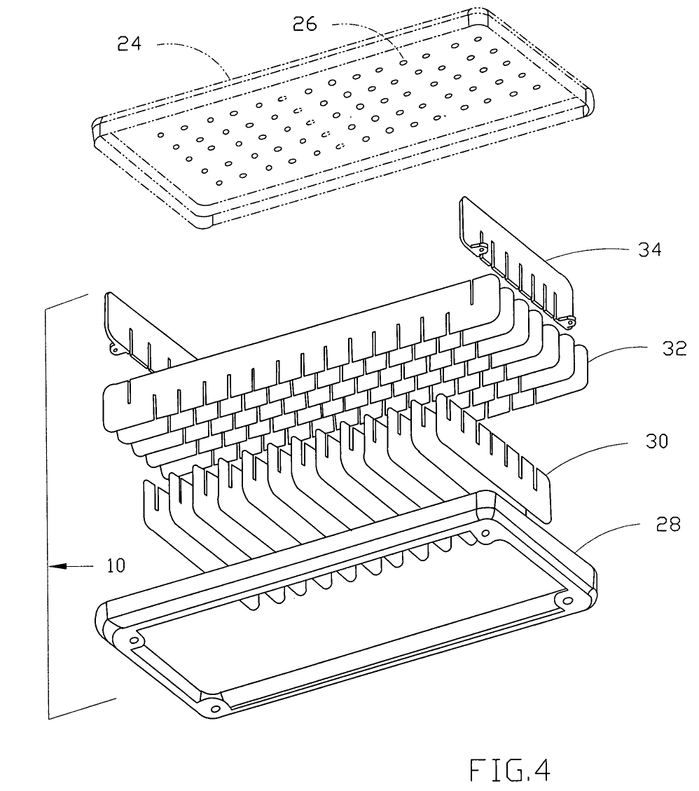

In order that the invention may be more fully understood, it will now be described by way of example with reference to the accompanying drawings. The improvement in the method of manufacture involves replacing the stamped sheet metal case as shown in FIG. 1 PRIOR ART or the molded plastic case as shown in FIG. 2 PRIOR ART with molded or extruded matrix as shown in FIG. 3 or the assembly of the interlocking members 30, 32 and 34 attached to outer wrapper 28 as shown in FIG. 4.

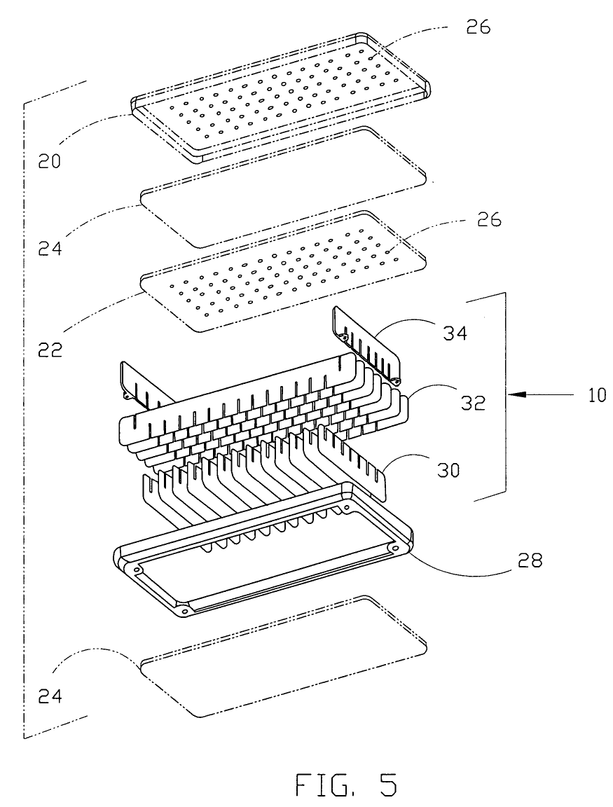

FIG. 3 illustrates an embodiment of sterilization case 10 that is formed by a molding or extrusion process. FIGS. 4 and 5 illustrate a second and preferred embodiment of sterilization case 10 formed by assembling egg crate type interlocking members 30, 32 and 34 and attaching that assembly inside outer wrap 28. The contours, not shown, of the underside of instruments 14 that are to be included in a particular surgery are cut into the top surface of the matrix members in both embodiments. All the material except the weight bearing portions of the matrix members and the required interconnecting webs is removed making these structures very lightweight while retaining substantial strength.

FIG. 4 shows an exploded perspective view of sterilization case 10 formed by placing as many bottom interlocking members 30 transverse to the long axis of the matrix as are required to provide sufficient length to cover instruments 14 needed for a particular surgery. A sufficient quantity of top interlocking members 32 are engaged over the bottom interlocking members 30, parallel to the long axis of the matrix to provide sufficient width to cover required instruments 14. Left and right hold down interlocking members 34 are engaged over the upward facing end slots of top members 32. The matrix assembly is then placed into outer wrapper 28 and held in place by fasteners through the mounting tabs at the ends of hold down members and into mounting tabs at the inside bottom corners of outer wrapper 28. The material and finish of the load bearing members 30, 32 and 34 can be varied as required within one matrix assembly. The matrix members in the interlocking embodiment are replaceable for addition of a new instrument or sequence of instruments within the case or outer wrapper and the matrix can be readily exchanged as it is mounted to the outer wrapper with a minimum of fasteners. FIG. 4 also shows, for illustrative purposes only in phantom lines, top enclosure 24 with its ingress ports 26.

Weep ports 16 required in the bottoms of pockets 12 of the molded plastic case 10 or the stamped sheet metal case 10 with brackets 18 are no longer required as the matrix concept is a series of through holes connected with a matrix web.

Instruments 14 are retained in sterilization case with matrix base 10 with a deformable member 22 shown in phantom lines in FIG. 5 for illustrative purposes with matching ports 26 to allow ingress of the sterilizing fluid, restrained by top enclosure 20 with matching ports 26. Top enclosure 20 can be fastened to outer wrapper 28 with conventional means during sterilization, storage and transport, being removed after the sterile gauze has been removed in the operating room.

Graphic outlines of the instrument can be placed on outer wrapper 24 adjacent to the pocket 12, on a separate plate, not shown, or on the under side of top enclosure 20 if present, to assist in identifying the proper tool for the appropriate stage of the procedure.

### OPERATION

A matrix of rectangular interlocking members is formed either by molding or extrusion as shown in FIG. 3 or assembly of rectangular interlocking egg crate type structures as shown in FIGS. 4 and 5 and is constructed of sufficient rectangular cross section and depth to accommodate all the instruments utilized in a given surgical procedure. The matrix then has material from the top of the interlocking members removed, leaving the weight bearing contours and the interconnecting webbing.

After case 10 is loaded with required instruments 14, deformable member 22 and top enclosure 20 are installed. To assist the surgical team in getting the correct instrument in the correct sequence, Graphic outlines of the proper placement within the matrix may be placed on a separate plate as cutouts, stamped on the under side of the cover or noted on the outer wrapper.

The assembly is subjected to an autoclave type environment where steam is pumped through top enclosure 20 and deformable member 22, around instruments 14 and out the bottom of the open matrix. After sterilization, the assembly is allowed to cool in a controlled environment and wrapped in a sterile gauze or muslin material and stored until required. The assembly is then transported to the operating room where it is unwrapped and top enclosure 20 and deformable member 22 removed and instruments 14 presented to the surgical team.

This sterilization case with matrix base 10 may also be used in a filtered case with no top enclosure 20 and open bottom, as a filtered case with filters 24 and holders mounted on the bottom of outer wrapper 28 and the under side of top enclosure 20, and with top enclosure 20 with filter 24 and a gasket as shown in FIG. 5.

The preceding descriptions are for illustrative purposes and are not intended to limit the scope of this invention. The scope of the invention should be determined by the appended claims rather than by the specific examples given. Manufacturing techniques described herein are well known to those skilled in the arts of mass production but have not been embraced for this particular application and hold forth significant benefits for improving the cost effectiveness of these critical and rapidly escalating costs of modern surgery.

1. In a sterilization tray assembly for sterilizing, transporting and storing surgical instruments comprising a top enclosure with a plurality of ports for allowing the ingress of a gaseous sterilant, a pocketed case, a means for locking said top enclosure to said case; cooperating deformable members fitting in said top enclosure for supporting and clamping said instruments in said pockets where said deformable member has matching ports to allow said sterilant to reach said instruments; and said tray assembly is wrapped in sterile gauze after sterilization for storage and transport,
the improvement comprising utilizing a matrix case fabrication process selected from either molding or extrusion, whereby said case is formed, without the sharp corners or burrs that are common with sheet metal stamping that might puncture or tear said sterile gauze, from lightweight metal that has a higher heat transfer coefficient than plastics that allows a faster drying time than is available with plastic molded trays.

2. In a sterilization tray assembly for sterilizing, transporting and storing surgical instruments as in claim 1 whereby said matrix contains as many elements as required for a specific surgery instrument set and the elements are as long and as deep as required to surround and support said instruments.

3. In a sterilization tray assembly for sterilizing, transporting and storing surgical instruments as in claim 1 whereby graphic outlines are placed on a separate plate as cut outs, on the underside of top enclosure and on the side of said outer wrapper depending on application.

4. In a sterilization tray assembly for sterilizing, transporting and storing surgical instruments as in claim 1 wherein said matrix case can be utilized in an application chosen from a group consisting of a filter case with said top enclosure with sterilant ingress ports, a top filter fastened to the under side of said top enclosure and a bottom filter attached to the bottom side of outer wraps; and in a filter case with no top enclosure or filters.

5. In a sterilization tray assembly for sterilizing, transporting and storing surgical instruments comprising a top enclosure with a plurality of ports for allowing the ingress of a gaseous sterilant, a pocketed tray, a means for locking said top enclosure to said case; cooperating deformable members fitting in said top enclosure for supporting and clamping said instruments in said pockets where said deformable member has matching ports to allow said sterilant to reach said instruments; and said tray assembly is wrapped in sterile gauze after sterilization for storage and transport,
the improvement comprising utilizing a matrix case fabrication process wherein the vertically oriented matrix is assembled from:
a plurality of bottom interlocking members that are rectangular in shape with a top surface, a length, a height, a thickness and front and back ends, a plurality of interlock slots that extend down from said top surface to half of said height, with said slot width being sufficiently wider than said member thickness to allow for a slip fit over said thickness of mating top interlocking members;
a plurality of top interlocking members that are rectangular in shape with a bottom surface, a top surface, a length, a height, a thickness, a left end and a right end, a plurality of interlock slots that extend up from said bottom surface to half of said height, spaced to interface with each of said plurality of bottom members, a slot on both the left and right ends that extend down from said top surface to half of said height, said slot width being sufficiently wider than said member thickness to allow for a slip fit of said interlock slots over said thickness of said mating top and bottom members;
left and right hold down interlocking members that are rectangular in shape with a bottom surface, a top surface, a length, a height, a thickness, a front end and a back end, a plurality of interlock slots that extend up from said bottom surface to half of said height, spaced to interface with each of said plurality of bottom members, mounting tabs formed at right angles from the vertical plane of said hold down members on said front and back ends at said bottom surface of said hold down members;
an outer wrap that is a rectangular box, open at the top and bottom, with the four walls of approximately the same height as said top, bottom and hold down interlocking members, with mounting tabs extending inward from the bottom said box walls in the four corners;
said bottom members are arranged wherein their length is transverse to the long axis of said outer wrapper and said top members are arranged perpendicularly to said bottom members and their mating slots engaged;
said hold down members are arranged perpendicularly to said top members at their left and right ends and their mating slots are engaged; and
the assembled matrix is disposed inside said outer wrapper and conventional fasteners are inserted between the four mounting tabs on said left and right hold down members and said mounting tabs in said four corners of outer wrapper holding said matrix securely in place.

6. In a sterilization tray assembly for sterilizing, transporting and storing surgical instruments as in claim 5 whereby said interlocking members are made from various materials with different finishes or coatings within one matrix assembly as might be required.

7. In a sterilization tray assembly for sterilizing, transporting and storing surgical instruments as in claim 5 whereby said matrix contains as many elements as required for a specific surgery instrument set and the elements are as long and as deep as required to surround and support said instruments.

8. In a sterilization tray assembly for sterilizing, transporting and storing surgical instruments as in claim 5whereby graphic outlines are placed on a separate plate as cut outs, on the underside of top enclosure and on the side of said outer wrapper depending on application.

9. In a sterilization tray assembly for sterilizing, transporting and storing surgical instruments as in claim 5 wherein said matrix case can be utilized in an application chosen from a group consisting of a filter case with said top enclosure with sterilant ingress ports, a top filter fastened to the under side of said top enclosure and a bottom filter attached to the bottom side of outer wraps; and in a filter case with no top enclosure or filters.
